(19) United States
(12) Patent Application Publication
(10) Pub. No.: US 2005/0113814 A1
(43) Pub. Date: May 26, 2005

(54) Apparatus and method for limiting the re-use of fiber optic, laser energy delivery devices

(76) Inventor: Marvin P. Loeb, Huntington Beach, CA (US)

Correspondence Address: Patent Law & Venture Group;Suite 150, 3140 Red Hill Avenue, Costa Mesa, CA, 92626-3440, US

(21) Appl. No.: 10/721,927

(22) Filed: Nov. 24, 2003

(51) Int. Cl. A61B 18/20

(52) U.S. Cl. 606/15; 606/16

(57) ABSTRACT

An apparatus for limiting the number of times a fiber-optic, laser energy delivery device can be reused consists of a sleeve attached about a portion of the body of an optical fiber and a handpiece and attached hollow cannula through which the portion of the optical fiber not encased in the sleeve extends. A compression means, whose aperture, when fully closed, has an inside diameter smaller than the outside diameter of the sleeve, but greater than the outside diameter of optical fibers generally available in the medical field, is provided.

### BACKGROUND OF THE INVENTION

### INCORPORATION BY REFERENCE

Applicant hereby incorporates herein by reference, any and all U.S. patents, U.S. patent applications, and other documents and printed matter cited or referred to in this application.

### FIELD OF THE INVENTION

This invention relates generally to optical fibers and their use and more particularly to a handpiece capable of mechanically supporting the optical fiber and holding it in place during use.

### DESCRIPTION OF PROBLEMS AND USE IN THE RELATED ART

Most commercial, medical grade optical fibers are supplied in lengths of three meters or longer. After use, the exterior buffer coating of the optical fiber can be clipped off and the fiber can be scored and cleaved to remove about five millimeters of the distal end of the optical fiber, which is often damaged by back scatter of laser energy during use and can emit laser energy at an aberrant angle. This allows an optical fiber to be re-used up to 50 or more times. Stresses encountered in such multiple uses can cause the optical fiber to crack at some point along its length and emit laser energy in an unpredictable direction, placing the patient and the operating room personnel at risk.

Excessive reuse of an optical fiber can be prevented by fixedly encasing all but a few millimeters or centimeters of the distal end of the optical fiber in a plastic or metal sleeve. The aperture of a compression device in the proximal end of a handpiece, through which the optical fiber must be inserted prior to use, when fully closed, must be smaller in diameter than the outside diameter of the plastic or metal sleeve fixedly attached to the optical fiber by heat shrinking, an adhesive, crimping and/or other means known in the art. However, the aperture of the compression device, when fully closed, must be larger than the outside diameter of the largest commonly available optical fibers used in the medical field, i.e., optical fibers whose core diameter is 0.1000 microns and whose outside diameter is about 1.7 mm. If the diameter of the plastic or metal sleeve is, for example, about 2.3 mm, and the aperture of the compression device closes to only about 2.0 mm, a commonly available optical fiber cannot be fixed in place and used with the handpiece.

A portion of an optical fiber extends distally from the distal end of the sleeve. A desired number of sections of the distal end portion of the optical fiber may be removed after each use, enabling it to be reused only a selected number of times.

The handpiece is designed so the sleeve cannot be advanced more than a desired distance into the handpiece. There are no stripping devices commonly available in the medical field able to strip or clip-off a portion of a sleeve with such a large outside diameter. The portion of the optical fiber extending from the distal end of the sleeve can contain circular, semi-circular or other markings at desired intervals, for example, at ½ cm intervals, enabling the operator to uniformly position the stripping device on the optical fiber and remove a uniform amount therefrom.

When the distal end face of a new, unused optical fiber is positioned at the distal end of a cannula which extends distally from a handpiece assembly provided for ease of use, the compression device in the proximal end of the handpiece can be closed about the sleeve, fixing the sleeve and the optical fiber in place and making the device ready for use. After use, the compression device is opened, the optical fiber is removed, a section of the distal end of the optical fiber is stripped, scored and cleaved, as aforesaid, and then the optical fiber and handpiece are cleaned and sterilized. When the distal end face of the optical fiber has been re-positioned at the distal end of the cannula, the compression device can engage the sleeve and fix it in place in the handpiece, as described above, making it ready for use.

After a desired number of strip, score and cleave procedures and uses, the sleeve about the optical fiber cannot be extended further into the handpiece assembly, no additional sections of the optical fiber are available to be stripped, scored and cleaved, and the optical fiber assembly must be discarded. If it is desired to limit the use of the optical fiber assembly to one use, the sleeve simply extends to the distal end of the interior of the handpiece, preventing reuse of the optical fiber assembly.

If the manufacturer wishes to limit the number of uses of a fiber optical assembly, for example, to ten, with each clipping procedure removing ½ cm of the buffer coat, the distal 5 cm of the optical fiber can have 10 markings on its buffer coat at ½ cm intervals. After the last available ½ cm of the optical fiber has been clipped, and the fiber has been scored, cleaved and the distal end face of the optical fiber has been positioned at the distal end of the cannula, as mentioned, the sleeve cannot be advanced farther into the handpiece. An optional red circle or other marking on the exterior of the sleeve can indicate to the user that the sleeve cannot be advanced further into the handpiece and the device, after its next use, must be discarded. Clearly, any other number of sections of the optical fiber can be marked for removal, and reuse of the device will be limited thereby.

Some stripping devices have a channel of a given length into which the fiber is inserted, which determines the length of the stripped-off buffer coat. Such channels usually admit about ½ cm of an optical fiber. If the channel of the stripping device supplied to the user with the optical fiber by the manufacturer is a different length, the intervals of the markings on the optical fiber may coincide with the length of the stripping device's channel.

The compression device can utilize a threaded nut which compresses a short length of flexible plastic tubing to a desired inside diameter, a metal tube with flanges or any other means known in the art, so long as the aperture of the compression device, when fully closed, is less than the outside diameter of the sleeve fastened about the optical fiber, but is larger than the outside diameter of optical fibers commonly available for use in the medical field.

Optical fiber assemblies that are used to transmit laser energy are typically supplied for medical use in lengths of three meters or longer. The quartz or fused silica core of the assembly is not affected by the transmission of a moderate amount of laser energy, but may crack or shatter if exposed to excessive laser energy over time and is bent during use. Backscatter of light energy from tissue has also been found to erode the distal end portion of the plastic buffer coating used to protect the glass cladding and the underlying core of the optical fiber. The glass cladding has an index of refraction significantly lower than that of the core, which causes light energy to be reflected back into the core as it passes along the length of the optical fiber assembly.

When the distal end portion of the buffer coating becomes eroded and the underlying glass cladding is damaged, laser energy can be emitted sidewise, or at a significantly divergent angle, rather than forwardly at a quite small divergent angle. Also, core fracture has been observed when the protective buffer coating is lost. Backscatter of light energy has also been found to melt and deform the distal end portion of the core of the optical fiber assembly. When the core becomes deformed or fractures, laser energy can be emitted in undesired directions. In such event, inadvertent damage to nerves, blood vessels and other tissues can result.

Typically, hospital or surgery center personnel strip or clip and remove about ½ cm of the buffer coating from the distal end of the optical fiber assembly and score and cleave the optical fiber at a point about 1 to 3 millimeters distally of the stripped, distal end, so as to create a flat distal end face. Conventional stripping, scoring and cleaving devices for this purpose are made by Micro Electronics, Inc. of Seeonk, Mass. As an example, optical fiber assemblies used in lithotripsy, that is, the fragmentation of urinary or biliary stones, are often stripped, scored, cleaved, sterilized and reused up to fifty or more times.

A related issue is unseen damage to the proximal end of an optical fiber assembly, which occurs when a portion of the influx of laser energy misses the core of the optical fiber and overheats the metal connector in which the optical fiber bundle is housed. This can cause failure of the optical fiber assembly at its proximal end, where it is coupled to a laser, as well as damage to the laser's optics or injury to operating room personnel.

It would be desirable to have a simple means to limit the number of times an optical fiber can be stripped, scored, cleaved and re-used to avoid these problems.

### SUMMARY OF THE INVENTION

The present invention teaches certain benefits in construction and use, which give rise to the objectives described below.

An apparatus for limiting the number of times a fiber-optic, laser energy delivery device can be reused consists of a sleeve attached about a portion of the body of an optical fiber and a handpiece and attached cannula through which the optical fiber extends. A compression means, whose aperture when fully closed has an inside diameter smaller than the outside diameter of the sleeve, but greater than the outside diameter of optical fibers generally available in the medical field, is provided.

A handpiece provides, at a distal end thereof, a metal cannula, and an axially oriented channel for accepting a sleeve of a fiber optical assembly therein. The channel may terminate at a shoulder within the channel or at the metal cannula, whose proximal end is of such diameter as to prevent advancement of the sleeve of the fiber optic assembly thereinto, while enabling the optical fiber to advance within the cannula. The channel has helical female threads within its proximal end. A compression cap provides male threads engaging the female threads of the channel. A means for gripping the sleeve when the cap is threadingly advanced into the channel is provided, so as to prevent axial motion of the sleeve/fiber optic assembly.

A primary objective of the present invention is to provide an apparatus and method of use of such apparatus that yields advantages not taught by the prior art.

Another objective is to provide such an invention capable of holding a fiber optic assembly in place during use.

A further objective is to provide an invention capable of preventing the removal of only a desired number of sections of the distal end of the fiber optic assembly.

A still further objective is to provide such an invention capable of being fed into a narrow channel and flexibly resuming its curved shape thereafter.

Other features and advantages of the present invention will become apparent from the following more detailed description, taken in conjunction with the accompanying drawings, which illustrate, by way of example, the principles of the invention.

### BRIEF DESCRIPTION OF THE DRAWINGS

The accompanying drawings illustrate the present invention. In such drawings:

FIG. 1 is a perspective view of a laser and an optical fiber assembly of the present invention;

FIG. 2 is a sectional view of a handpiece assembly thereof FIG. 3 is a sectional view of a compression element thereof;

FIG. 4 is a sectional view thereof showing an alternate compression element; and

FIGS. 5 and 6 are sectional views thereof.

### DETAILED DESCRIPTION OF THE INVENTION

The above described drawing figures illustrate the invention in at least one of its preferred embodiments, which is further defined in detail in the following description. Those having ordinary skill in the art may be able to make alterations and modifications in the present invention without departing from its spirit and scope. Therefore, it must be understood that the illustrated embodiments have been set forth only for the purposes of example and that they should not be taken as limiting the invention as defined in the following.

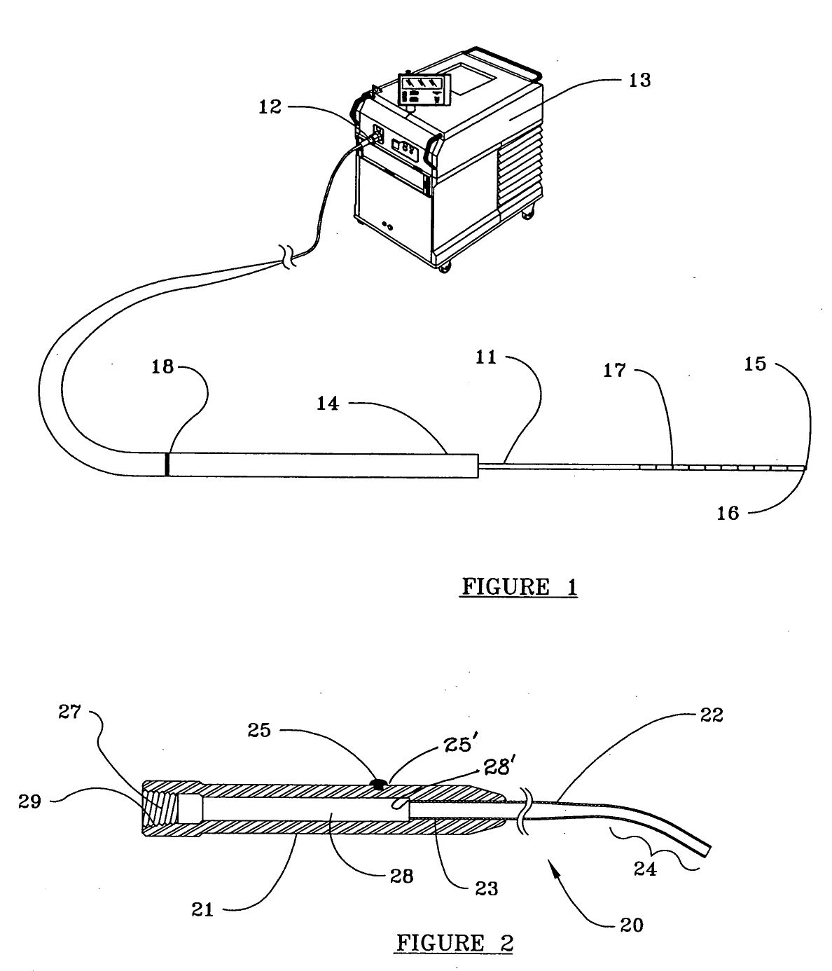

As seen in FIG. 1, the proximal end of optical fiber 11 is fixedly attached within connector 12 and optically coupled to laser 13. Plastic or metal sleeve 14, which preferably has an outside diameter of at least about 2.0 mm, is attached by an adhesive, heat shrinking, crimping or other means, to a portion of the body of optical fiber 11, preferably extending from connector 12 to the point at which optical fiber 11 emerges from the distal end of sleeve 14. Core 15 of optical fiber 11 extends distally, about 1 to 3 mm, from the distal end of a buffer coating 16.

The distal end portion of optical fiber 11 has markings 17 at desired intervals, preferably about ½ cm apart, around the buffer coating 16 of optical fiber 11 to assist the user in the stripping (clipping), scoring and cleaving process. Optional circle marking 18 about sleeve 14 indicates to the user the point at which sleeve 14 cannot be further advanced into a handpiece assembly (20), requiring the sleeve/fiber optic assembly to be discarded.

FIG. 2 illustrates handpiece assembly component 20 of the present invention. Handpiece assembly 20 consists of handpiece 21, within which the proximal end portion 23 of metal or plastic cannula 22 is fixedly attached by adhesive 23. Cannula 22 extends distally from the handpiece 21. Optionally, the distal end portion 24 of cannula 22 may have a larger or smaller outside diameter than the proximal portion of cannula 22 and may have a bend as shown. A button 25, which may be fixedly attached by an adhesive or pressure fitted in recess 25′ in handpiece 21, may be disposed on the side of handpiece 21 at a position opposite the direction of the bend in the distal end portion 24 of cannula 22. While a 30° bend in distal end portion 24 of cannula 22 is illustrated, any bend angle may be used, and cannula 22 may also be straight, in which case button 25 is eliminated.

Optionally, distal end portion 24 of cannula 22 can be made of a super-elastic memory metal, such as Nitinol®, an alloy of titanium and nickel. Such super-elastic memory metals are sold by Memry Corporation of Bethel, Conn. If distal end portion 24 is made of such a memory metal, it can be heat treated to retain (remember) a desired bend or shape configuration. Thereafter, even if straightened-out a number of times, as for instance while passing through a channel of a rigid endoscope or a constraining metal tube, distal end portion 24 of cannula 22 will return to its heat treated bend configuration.

Handpiece 21 has opening 27 in its proximal end, which is larger in diameter and is in communication with channel 28, which extends through the body of handpiece 21. The diameter of channel 28 is slightly larger than the outside diameter of sleeve 14 of FIG. 1 and terminates at shoulder 28′ which prevents sleeve 14 from being further advanced within channel 28 of handpiece 21.

Alternatively, the inside diameter of cannula 22 can be smaller in diameter than the outside diameter of sleeve 14 and prevents sleeve 14 from being advanced into cannula 22.

Opening 27 in the proximal end of handpiece 21 has helical threads 29 about its inner surface, which threads 29 become smaller in diameter as they progress distally from the proximal end of opening 27, i.e., create an inwardly tapered or convergent opening.

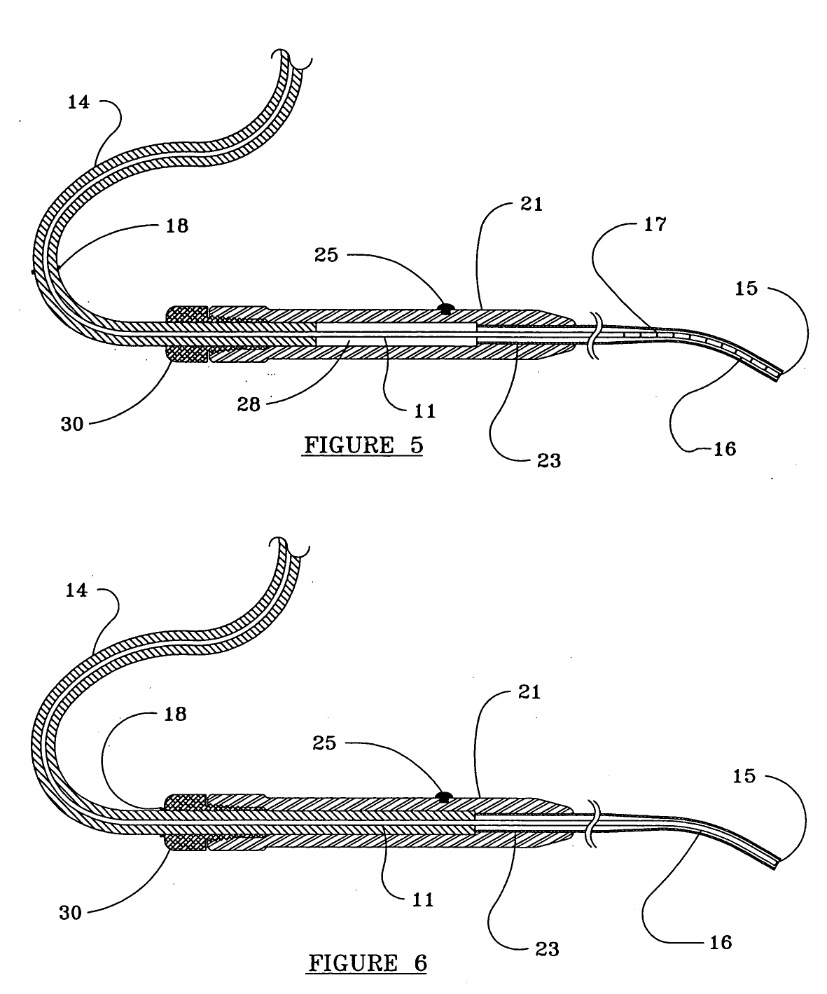

FIG. 3 illustrates a compression cap 30 for use with handpiece assembly 20 of FIG. 2, but cap 30 is not shown in FIG. 2. Compression cap 30 has threads 31 of uniform outside diameter on the exterior of its proximal end portion 33. Threads 31 of compression cap 30 engage decreasing diameter threads 29 within opening 27 in the proximal end of handpiece 21. As knob 32 is turned clockwise, the decreasing diameter of threads 29 causes metal or plastic flanges 33 in the distal end of compression device 30 to close and grasp sleeve 14 of FIG. 1. This is clearly shown in FIGS. 5 and 6. However, when knob 32 is fully screwed into threads 29, flanges 33 close not more tightly than to an inside diameter that is larger than the outside diameter of the largest of the commonly available optical fibers.

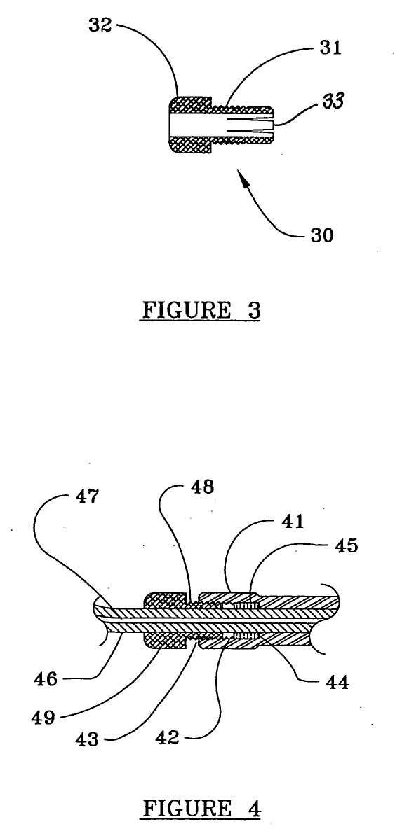

FIG. 4 illustrates an alternate embodiment of the compression device of FIG. 3. As seen in FIG. 4, an alternately configured handpiece 41 has helical threads 42 of uniform inside diameter within the inner surface of opening 43 in the proximal end of handpiece 41. The distal end 44 of opening 43 constrains the distal end of flexible, plastic tube 45, through which sleeve 46, fixedly encasing optical fiber 47 extends. Threads 48 on the exterior of the distal end portion of knob 49 engage threads 42 of handpiece 41. As shown, knob 49 is not fully screwed into opening 43 in handpiece 41. When knob 49 is screwed clockwise further into opening 43 in the proximal end of handpiece 41, knob 49 compresses the length of flexible plastic tube 45, causing it to expand in diameter and engage the exterior of sleeve 46, removably fixing it in place.

As illustrated in FIG. 5, the components of FIGS. 1-3 have been assembled for their first use. The distal end face of optical fiber 11 is positioned at the distal end of cannula 22, and the distal end of sleeve 14 is positioned a selected distance proximally from shoulder 28′ of channel 28. All of the markings 17 about the distal end portion of optical fiber 11 are within cannula 22.

As seen in FIG. 6, after all of the marked sections of optical fiber 11 have been removed, the distal end face of optical fiber 11 is at the distal end of cannula 22, and the distal end of sleeve 14 has reached shoulder 28′ of channel 28 and cannot be advanced further. Optional marking 18 on sleeve 14 has reached the proximal end of knob 32, indicating to the user that the last use of the device has been reached, after which it must be discarded.

While not illustrated in the figures, the distal end of cannula 22 can be flared outwardly, to prevent its being damaged by backscatter of laser energy reflected from tissue. Also, while not illustrated in the figures, a fluid port can be provided in handpiece 21 or cannula 22 to enable a gas, such as carbon dioxide, or a liquid such as saline, to be infused through the cannula 22. Infusing a gas displaces irrigation liquids and blood from the target tissue, avoiding the loss of energy in vaporizing the intervening liquid. Alternatively, infusing a liquid cools the optical fiber and the targeted tissue.

The words used in this specification to describe the invention and its various embodiments are to be understood not only in the sense of their commonly defined meanings, but to include by special definition in this specification: structure, material or acts beyond the scope of the commonly defined meanings. Thus if an element can be understood in the context of this specification as including more than one meaning, then its use must be understood as being generic to all possible meanings supported by the specification and by the word or words describing the element.

The definitions of the words or elements of this described invention and its various embodiments are, therefore, defined in this specification to include not only the combination of elements which are literally set forth, but all equivalent structure, material or acts for performing substantially the same function in substantially the same way to obtain substantially the same result. In this sense it is therefore contemplated that an equivalent substitution of two or more elements may be made for any one of the elements in the invention and its various embodiments below or that a single element may be substituted for two or more elements in a claim.

Changes from the claimed subject matter as viewed by a person with ordinary skill in the art, now known or later devised, are expressly contemplated as being equivalents within the scope of the invention and its various embodiments. Therefore, obvious substitutions now or later known to one with ordinary skill in the art are defined to be within the scope of the defined elements. The invention and its various embodiments are thus to be understood to include what is specifically illustrated and described above, what is conceptually equivalent, what can be obviously substituted, and also what essentially incorporates the essential idea of the invention.

While the invention has been described with reference to at least one preferred embodiment, it is to be clearly understood by those skilled in the art that the invention is not limited thereto. Rather, the scope of the invention is to be interpreted only in conjunction with the appended claims and it is made clear, here, that the inventor believes that the claimed subject matter is the invention.

1. An apparatus comprising: a handpiece providing, at a distal end thereof, an axial cannula, the handpiece further providing an axial channel for accepting a sleeve of an optical fiber, the axial channel terminating at the axial cannula; the axial cannula of such size as to receive advancement of the optical fiber therewithin, while blocking advancement of the sleeve; a hollow compression cap engaged within a proximal end of the axial channel; the compression cap providing a means for gripping the sleeve upon advancement of the compression cap into the channel, so as to selectively prevent axial motion of the sleeve and optical fiber.

2. The apparatus of claim 1 wherein the compression cap is threaded into the channel by converging threads; the gripping means comprising axially oriented fingers, arranged and positioned to close on the sleeve upon advancement of the compression cap into the channel.

3. The apparatus of claim 2 wherein the fingers are enabled for closing only to a diameter that is larger than a selected commercially available medical optical fiber.

4. The apparatus of claim 1 wherein the cannula is made, at least partially, of a super-elastic memory metal and is axially non-linear.

5. The apparatus of claim 4 wherein the super-elastic memory metal is Nitinol®.

6. The apparatus of claim 1 further comprising a compressible tube positioned and restrained at a proximal end of the channel and axially compressible by advancement of the compression cap, thereby providing a gripping force on the sleeve.

7. An apparatus comprising in combination: a handpiece providing, at a distal end thereof, an axial cannula, the handpiece further providing an axial channel terminating in a shoulder; a sleeve, coaxially fixed about an optical fiber, set axially within the axial channel and terminating at the shoulder; the axial cannula of such size as to receive advancement of the optical fiber therewithin, while the sleeve is blocked by the shoulder; a hollow compression cap engaged within a proximal end of the axial channel; the compression cap providing a means for gripping the sleeve upon advancement of the compression cap into the channel, so as to selectively prevent axial motion of the sleeve and optical fiber.

8. The apparatus of claim 7 wherein the compression cap is threaded into the channel by converging threads; the gripping means comprising axially oriented fingers, such that advancement of the compression cap into the channel closes the fingers onto the sleeve for thereby gripping the sleeve.

9. The apparatus of claim 7 wherein the fingers are enabled for closing only to a diameter that is larger than a selected commercially available medical optical fiber.

10. The apparatus of claim 7 wherein the cannula is made, at least partially, of a super-elastic memory metal and is non-linear.

11. The apparatus of claim 10 wherein the super-elastic memory metal is Nitinol®.

12. The apparatus of claim 7 further comprising a compressible tube positioned and restrained at a proximal end of the channel and axially compressible by advancement of the compression cap, thereby providing a gripping force on the sleeve.

13. A method of limiting the reuse of an optical fiber comprising the steps of: providing, at a distal end of an axial channel in a handpiece, an axial cannula; setting a sleeve coaxially about an optical fiber and within the axial channel; forming the sleeve of a size larger than the axial cannula; advancing the optical fiber within the axial cannula; engaging a hollow compression cap within a proximal end of the axial channel; providing a means for gripping the sleeve upon advancement of the compression cap into the channel, so as to selectively prevent axial motion of the sleeve and optical fiber.

14. The method of claim 13 further comprising threading the compression cap into the channel through converging threads; tightening axially oriented fingers during advancement of the compression cap into the channel to thereby close the fingers onto the sleeve for gripping thereof.

15. The method of claim 13 further comprising positioning a compressible tube restrained at a proximal end of the channel, and advancing the compression cap axially thereby compressing the tube to produce a gripping force on the sleeve.

16. A method of limiting the reuse of an optical fiber comprising the steps of: providing a handpiece having, at a distal end thereof, an axial cannula, the handpiece further providing an axial channel terminating in a shoulder; placing a sleeve, coaxially fixed about an optical fiber, axially within the axial channel and terminating the sleeve at the shoulder; advancing the optical fiber within the axial cannula while blocking advancement of the sleeve by the shoulder; engaging a hollow compression cap within a proximal end of the axial channel; providing the compression cap with a means for gripping the sleeve upon advancement of the compression cap into the channel, so as to selectively prevent axial motion of the sleeve and optical fiber.
